(19) United States
(12) Patent Application Publication
(10) Pub. No.: US 2002/0142312 A1
(43) Pub. Date: Oct. 3, 2002

(54) Polymorphism of the human serotonin 1B receptor gene, diagnostic methods and methods of treatment based thereon

(76) Inventors: Tessa Cigler, Charlotte, NC (US); Karl Steven LaForge, New York, NY (US); Mary Jeanne Kreek, New York, NY (US)

Correspondence Address: KLAUBER & JACKSON, 411 HACKENSACK AVENUE, HACKENSACK, NJ, 07601

(21) Appl. No.: 09/855,991

(22) Filed: May 15, 2001

Related U.S. Application Data
(60) Provisional application No. 60/204,169, filed on May 15, 2000.

(51) Int. Cl. C12Q 1/68; C07H 21/04; C07K 14/705; C12P 21/02; C12N 5/06

(52) U.S. Cl. 435/6; 435/69.1; 435/320.1; 435/325; 530/350; 536/23.5

(57) ABSTRACT

Provided herein is a variant allele of a gene encoding a serotonin 1B receptor, A−161T, along with cloning vectors for replicating such variant alleles, and expressing vectors for expressing the variant alleles to identify alterations in the expression of serotonin 1B receptors, for identifying individuals predisposed to addictive, neurologic or psychiatric diseases.

### CROSS-REFERENCE TO RELATED APPLICATION

This application claims priority to Provisional Application Serial No. 60/204,169, filed May 15, 2000.

### GOVERNMENTAL SUPPORT

This invention was made government support under the U.S. Public Health Service, National Institutes of Health, National Institute of Drug Addiction, grants DA05130 and DA00049. The Government has certain rights in the invention.

### FIELD OF THE INVENTION

This invention relates generally to a polymorphism of the human serotonin 1B receptor gene, useful for methods of diagnosing various susceptibilities using such polymorphism and determining treatment for certain diseases based upon the presence of the specific polymorphism.

### BACKGROUND OF THE INVENTION

Located primarily in peripheral and central neurons, serotonin (5-hydroxytryptamine, 5-HT) receptors appear to be involved in the depolarization of peripheral neurons, and the modulation of pain and the emesis reflex. Serotonin is implicated in the etiology or treatment of various disorders, including anxiety, depression, obsessive-compulsive disorder, schizophrenia, stroke, obesity, pain, hypertension, vascular disorders, migraine, headaches, and nausea. 5-HT is synthesized in situ from tryptophan through the actions of the enzymes tryptophan hydroxylase and aromatic L-amino acid decarboxylase. Both dietary and endogenous 5-HT are rapidly metabolized and inactivated by monoamine oxidase and aldehyde dehydrogenase to the major metabolite, 5-hydroxyindoleacetic acid (5-HIAA). The major mechanism by which the action of serotonin is terminated is by reuptake into presynaptic membranes. After 5-HT acts on its various postsynaptic receptors, it is removed from the synaptic cleft back into the nerve terminal through an uptake mechanism involving a specific membrane transporter in a manner similar to that of other biogenic amines. Agents that selectively inhibit this uptake increase the concentration of 5-HT at the postsynaptic receptors and have been found to be quite useful in treating various psychiatric disorders, particularly depression. Selective 5-HT reuptake inhibitors (SSRIs) have been investigated as potential antidepressants with the anticipation that these agents would possess fewer side effects, such as anticholinergic actions and cardiotoxicity, and would be less likely to cause sedation and weight gain.

Therapeutic use of agents acting at this site include treatment for migraine, anxiety, substance abuse, and cognitive and psychotic disorders. There are at least four populations of receptors for serotonin: 5-HT1, 5-HT2, 5-HT3, and 5-HT4. Recent cloning studies suggest the existence of 5-HT5, 5-HT6, and 5-HT7 subtypes as well. In addition at least five distinct subtypes of the 5-HT2 and three subtypes of the 5-HT3 receptors exist. Largely due to the complexity of these multiple subtypes, the physiological function of each receptor subtype has not been fully established. With the exception of the 5-HT3 receptor, which is a ligand-gated ion channel related to NMDA, GABA and nicotinic receptors, all of the 5-HT receptor subtypes belong to the group of G-protein linked receptors.

In addition to treating depression, several other potential therapeutic applications for SSRIs have been investigated which link serotonin receptor-related defects with various conditions and diseases . They include treatment of Alzheimer's disease; modulation of aggressive behavior; treatment of premenstrual syndrome, diabetic neuropathy, and chronic pain; and suppression of alcohol intake. Also significant is the observation that 5-HT reduces food consumption by increasing meal-induced satiety and reducing hunger, thus, there is interest in the possible use of SSRIs in the treatment of obesity.

It is towards the identification of a new single-nucleotide polymorphism (SNP) in a human serotonin receptor gene, and the diagnostic and therapeutic uses of such a polymorphism, that the present invention is directed.

The citation of any reference herein should not be construed as an admission that such reference is available as “Prior Art” to the instant application.

### SUMMARY OF THE INVENTION

There is provided, in accordance with the present invention, a heretofore unknown polymorphism of the human serotonin 1B receptor gene, a change from adenosine (A) to thymidine (T) at position −161 (minus 161). The invention is further directed to the use of this polymorphism and means for its detection in mapping the locus of the human serotonin 1B receptor gene, in determining susceptibility to various conditions and diseases which are solely or in part under the regulation of the serotonin 1B receptor gene, and in identifying therapeutic regimens suited for individuals with the polymorphism. Such conditions and diseases include but are not limited to various addictive diseases, neurologic and psychiatric diseases. The sequence of the gene wild-type serotonin 1B receptor containing the A−161T SNP is set forth in SEQ ID No:2.

The A−161T SNP has been found in about 20% of a particular human population, among which several individuals were found to be homozygous for this SNP.

Diseases and conditions include susceptibility to addictive, neurologic or psychiatric conditions, illnesses or diseases, and determining a therapeutically effective amount of therapeutic agent directed to the particular condition, to administer to a subject suffering from such conditions, and selecting an appropriate therapeutic agent and a therapeutically effective amount of such an agent to administer to a subject suffering from a disease or disorder related thereto.

The present invention extends to the heretofore unknown polymorphism of the human serotonin 1B receptor gene that can serve as genetic markers to map the locus of the human serotonin 1B receptor gene.

The present invention further extends to DNA sequences of heretofore unknown isolated nucleic acid molecules which encode a human serotonin 1B receptor, wherein the DNA sequence optionally includes a combination of presently known polymorphisms together with the polymorphism of the human serotonin 1B receptor discovered by Applicants.

The present invention further extends to diagnostic methods to determine a subject's increased or decreased susceptibility to addictive and other diseases. With the results of such methods, targeted prevention methods, early therapeutic intervention, and improved chronic treatment to opioid addiction are set forth herein and encompassed by the present invention. In addition, attending medical professionals armed with the results of such diagnostic methods can determine whether administration of opioid analgesics is appropriate or whether non-opioid derived analgesics should be administered to the subject. Furthermore, appropriate choice and type of analgesic to treat a subject's pain or other condition can be made.

Furthermore, the present invention extends to an isolated variant allele of a human serotonin 1B receptor gene as set forth above, which is detectably labeled. Numerous detectable labels have applications in the present invention, such as radioactive elements, chemicals that fluoresce, or enzymes, to name only a few.

The present invention further extends to an isolated nucleic acid molecule hybridizable under standard hybridization conditions to an isolated variant allele of the human serotonin 1B receptor gene, wherein the predominant or “most common” allele of a human serotonin 1B receptor gene found in the population comprises a DNA sequence of SEQ ID NO:1, and a variant allele of the present invention comprises a DNA sequence having a variation in SEQ ID NO:1, wherein the variation comprises A−161T.

Moreover, the present invention extends to an isolated nucleic acid molecule hybridizable under standard hybridization conditions to an isolated variant allele of the human serotonin 1B receptor gene, wherein the predominant or “most common” allele of the human serotonin 1B receptor gene found in the population comprises a DNA sequence of SEQ ID NO:1, and a variant allele of the present invention comprises a DNA sequence having a variation in SEQ ID NO:1, wherein the variation comprises A-161T, wherein the isolated nucleic acid molecule is detectably labeled. Examples of detectable labels that have applications in this embodiment of the present invention are described above.

In addition, the present invention extends to cloning vectors that can be used to clone copies of a variant allele of the human serotonin 1B receptor gene of the present invention. For example, the present invention extends to a cloning vector comprising an isolated variant allele of a human serotonin 1B receptor gene and an origin of replication, wherein the predominant or “most common” allele of the human serotonin 1B receptor gene found in the population comprises a DNA sequence of SEQ ID NO:1, and the variant allele of the present invention comprises a DNA sequence having a variation in SEQ ID NO:1, wherein the variation comprises A−161T.

In another embodiment, the present invention extends to a cloning vector comprising an isolated nucleic acid molecule hybridizable under standard hybridization conditions to an isolated variant allele of the human serotonin 1B receptor gene, and an origin of replication, wherein the predominant or “most common” allele of the human serotonin 1B receptor gene found in the population comprises a DNA sequence of SEQ ID NO:1, and a variant allele of the present invention comprises a DNA sequence having a variation in SEQ ID NO:1, wherein the variation comprises A−161T.

Numerous cloning vectors have applications in the present invention. For example, a cloning vector having applications in the present invention includes E. coli, bacteriophages such as lambda derivatives, plasmids such as pBR322 derivatives, and pUC plasmid derivatives such as pGEX vectors or pmal-c or pFLAG, to name only a few.

Furthermore, the present invention extends to an isolated variant allele of the human serotonin 1B receptor gene, wherein the predominant or “most common” allele of the human serotonin 1B receptor gene comprises a DNA sequence of SEQ ID NO:1, and a variant allele of the present invention comprises a DNA sequence having at least two variations in SEQ ID No: 1, one being A−161T. The other variation may be any other polymorphisms in the serotonin 1B receptor gene, including but not limited to those already known.

The present invention further extends to an isolated variant allele of a human serotonin 1B receptor gone comprising a DNA sequence having at least two variations in SEQ ID NO:1, as stated above, which is detectably labeled. Examples of detectable labels having applications in this embodiment include, but are not limited to, a radioactive element, a chemical that fluoresces, or an enzyme.

The present invention further extends to an isolated nucleic acid molecule hybridizable under standard hybridization conditions to an isolated variant allele of a human serotonin 1B receptor gene, wherein the predominant or “most common” allele of the human serotonin 1B receptor gene comprises a DNA sequence of SEQ ID NO:1, and a variant allele of the present invention comprises a DNA sequence having at least two variations in SEQ ID NO:1, wherein one variation is A−161T.

Naturally, the present invention extends to a detectably labeled isolated nucleic acid molecule hybridizable under standard hybridization conditions to an isolated variant allele of a human serotonin 1B receptor comprising a DNA sequence having at least two variations in SEQ ID No: 1, one being A−161T. The other variation may be any other polymorphisms in the serotonin 1B receptor gene, including but not limited to those already known.

Examples of detectable labels having applications in this embodiment of the invention include, but are not limited to, a radioactive element, a chemical which fluoresces, or an enzyme.

Furthermore, the present invention extends to a cloning vector comprising an isolated variant allele of a human serotonin 1B receptor gene and an origin of replication, wherein the predominant or “most common” allele of the human serotonin 1B receptor gene present in the population comprises a DNA sequence of SEQ ID NO:1, and a variant allele of the present invention comprises a DNA sequence having at least two variations in SEQ ID NO:1, one being A−161T. The other variation may be any other polymorphisms in the serotonin 1B receptor gene, including but not limited to those already known.

In addition, the present invention extends to a cloning vector comprising an isolated nucleic acid molecule hybridizable under standard hybridization conditions to a variant allele of a human serotonin 1B receptor and an origin of replication, wherein the variant allele comprises a DNA sequence having at least two variations in SEQ ID NO:1, one variation being A−161T. The other variation may be any other polymorphisms in the serotonin 1B receptor gene, including but not limited to those already known.

Numerous cloning vectors have applications in this embodiment of the present invention. Examples of such vectors include, but are not limited to, E. coli, bacteriophages, such as lambda derivatives, plasmids such as pBR322 derivatives, and pUC plasmid derivatives such as pGEX vectors or pmal-c or pFLAG, to name only a few.

Accordingly, the present invention extends to a method for determining a susceptibility in a subject to at least one addictive disease, comprising the steps of removing a bodily sample comprising a first and second allele of a human serotonin 1B receptor gene from the subject, and determining whether the first allele comprises the human serotonin 1B receptor gene comprising a DNA sequence having at least one variation in SEQ ID NO:1, wherein the at least one variation is A−161T.

The presence of this variation in the human serotonin 1B receptor gene of the first allele is expected to be indicative of the subject's susceptibility to at least one addictive disease relative to the susceptibility of a standard to at least one addictive disease, wherein the standard comprises a first allele comprising a human serotonin 1B receptor gene having a DNA sequence of SEQ ID NO:1.

Another embodiment of the method for determining a susceptibility in the subject to at least one addictive disease, as described above, comprises the further step of determining whether the second allele of the bodily sample of the subject comprises the human serotonin 1B receptor gene comprising a DNA sequence having at least one variation in SEQ ID NO:1, wherein the at least one variation comprises A−161T.

The presence of at least one variation the second allele of the bodily sample is expected to be indicative of the subject's susceptibility to at least one addictive disease relative to a standard in which both alleles of the human serotonin 1B receptor gene comprise a DNA sequence of SEQ ID NO:1.

In particular, the presence of an A−161T variation in the DNA sequence of the human serotonin 1B receptor gene of the first and/or second alleles in the bodily sample from the subject is expected to be indicative of altered susceptibility of the subject to at least one addictive disease relative to the standard.

Moreover, the presence of a A−161T variation in the DNA sequence of the human serotonin 1B receptor gene of the first and/or second alleles in the bodily sample from the subject is expected to be indicative of an altered susceptibility of the subject to at least one addictive disease relative to the susceptibility of the standard to at least one addictive disease wherein both alleles of the standard comprise the human serotonin 1B receptor gene comprising a DNA sequence of SEQ ID NO:1. Examples of at least one addictive disease includes, but is not limited to opioid addiction, cocaine addiction or addiction to other psychostimulants, nicotine addiction, barbiturate or sedative hypnotic addiction, anxiolytic addiction, or alcohol addiction.

Furthermore, the present invention extends to a method for determining a susceptibility to pain in a subject relative to susceptibility to pain in a standard, comprising the steps of removing a bodily sample comprising a first and second allele of the human serotonin 1B receptor gene from the subject, and determining whether the first allele comprises the human serotonin 1B receptor gene comprising a DNA sequence having at least one variation in SEQ ID NO:1, wherein the variation comprises A−161T. The presence of at least one variation in the human serotonin 1B receptor gene of the first allele is expected to be indicative of a decreased or increased susceptibility to pain in the subject relative to susceptibility to pain in the standard, wherein the first allele of the standard comprises the human serotonin 1B receptor gene comprising a DNA sequence of SEQ ID NO:1.

Moreover, a method for determining a susceptibility to an addictive, neurologic or psychiatric condition or disease in a subject may further comprise the step of determining whether the first and/or second allele comprises the human serotonin 1B receptor gene comprising a DNA sequence having at least one variation in SEQ ID NO:1, wherein the variation comprises A−161T.

The presence of at least one variation in the human serotonin 1B receptor gene of the second allele of the bodily sample from the subject is expected to be indicative of an increased or decreased susceptibility to an addictive, neurologic or psychiatric condition or disease in the subject relative to the susceptibility to an addictive, neurologic or psychiatric condition or disease in the standard, wherein the second allele in the standard comprises the human serotonin 1B receptor gene comprising a DNA sequence of SEQ ID NO:1.

At least one variation in the human serotonin 1B receptor gene of the first and/or second allele of the bodily sample taken from the subject, wherein the variation comprises A−161T, is expected to be indicative of an altered susceptibility to an addictive, neurologic or psychiatric condition or disease in the subject relative susceptibility of pain in the standard, wherein the human serotonin 1B receptor gene of the first and/or second allele of the standard comprises a DNA sequence of SEQ ID NO:1.

Furthermore, the presence of at least one variation comprising A−161T in the human serotonin 1B receptor gene of the first and/or second allele of the bodily sample from the subject is expected to be indicative of an altered susceptibility to an addictive, neurologic or psychiatric condition or disease in the subject relative to the susceptibility to an addictive, neurologic or psychiatric condition or disease in the standard, wherein the first and/or second allele of the standard comprise the human serotonin 1B receptor gene comprising a DNA sequence of SEQ ID NO:1.

Once a susceptibility to an addictive, neurologic or psychiatric condition or disease in the subject has been determined, it is possible for attending medical professionals treating the subject to accordingly administer an appropriate, or therapeutically effective amount of pain reliever in order to induce analgesia in the subject. Administration of an appropriate amount is very important. For example, should an inappropriate amount of pain reliever be administered, the subject may not experience analgesia, and may be exposed to potentially deleterious side effects of the pain reliever, such as induction of addiction to the pain reliever, brain damage, or death.

Consequently, the present invention extends to a method for determining a therapeutically-effective amount of pain reliever to administer to a subject in order to induce analgesia in the subject relative to a therapeutically effective amount of the pain reliever to administer to a standard in order to induce analgesia in the standard, wherein the method comprises determining a susceptibility to an addictive, neurologic or psychiatric condition or disease in the subject relative to susceptibility to an addictive, neurologic or psychiatric condition or disease in the standard. The susceptibility of pain in the subject is expected to be indicative of the therapeutically effective amount of the pain reliever to administer to the subject to induce analgesia in the subject relative to the amount of the pain reliever to administer to the standard to induce analgesia in the standard.

Hence, the present invention extends to a method for determining a therapeutically effective amount of pain reliever to administer to a subject in order to induce analgesia in the subject relative to a therapeutically effective amount of the pain reliever to administer to a standard in order to induce analgesia in the standard wherein the method comprises the steps of removing a bodily sample comprising a first and second allele of the human serotonin 1B receptor gene from the subject, and determining whether the first allele comprises the human serotonin 1B receptor gene comprising a DNA sequence having at least one variation in SEQ ID NO:1, wherein the variation comprises A−161T.

The presence of at least one variation in the human serotonin 1B receptor gene of the first allele from the bodily sample is expected to be indicative of the therapeutically effective amount of pain reliever to administer to the subject to induce analgesia in the subject relative to the therapeutically effective amount of pain reliever to administer to the standard to induce analgesia in the standard, wherein the standard comprises a first allele comprising the human serotonin 1B receptor gene comprising a DNA sequence of SEQ ID NO:1.

Moreover, the present invention further extends to a method for determining a therapeutically effective amount of pain reliever to administer to a subject in order to induce analgesia in the subject relative to a therapeutically effective amount of pain reliever to administer to a standard to induce analgesia therein, further comprising the steps of removing a bodily sample comprising a first and second allele comprising the human serotonin 1B receptor gene from the subject, and determining whether the second allele of the bodily sample comprises the human serotonin 1B receptor gene comprising a DNA sequence comprising at least one variation in SEQ ID NO:1, wherein the variation comprises A−161T.

The presence of at least one variation in the human serotonin 1B receptor gene of the first and/or second allele of the bodily sample is expected to be indicative of the therapeutically effective amount of pain reliever to administer to the subject to induce analgesia therein relative to the amount of pain reliever to administer to a standard to induce analgesia therein, wherein the first and second alleles of the standard comprise the human serotonin 1B receptor gene comprising a DNA sequence of SEQ ID NO:1.

More particularly, a variation of the human serotonin 1B receptor gene of the first and/or second allele from the bodily sample taken from the subject, comprising a DNA sequence comprising a variation in SEQ ID NO:1, wherein the variation comprises A−161T, is expected to be indicative of an altered susceptibility to an addictive, neurologic or psychiatric condition or disease in the subject relative susceptibility of pain in the standard. Consequently, the subject requires a different therapeutically effective amount of pain reliever in order to induce analgesia therein relative to the therapeutically effective amount of pain reliever needed to induce analgesia in the standard.

Furthermore, the present invention extends to a method for determining a therapeutically effective amount of a therapeutic agent for treating at least one addictive disease to administer to a subject suffering from at least one addictive disease, relative to a therapeutically effective amount of the therapeutic agent to administer to a standard suffering from the at least one addictive disease. As a result, the dosage of therapeutic agent administered to an addict can be “tailored” to the addict's needs based upon the addict's genotype. An example of such a method comprises the steps of removing a bodily sample from the subject, wherein the bodily sample comprises a first and second allele of the human serotonin 1B receptor gene, and determining whether the first allele comprises a DNA sequence having at least one variation in SEQ ID NO:1, wherein the variation comprises A−161T.

The presence of the at least one variation in the human serotonin 1B receptor gene of the first allele in the bodily sample from the subject is related to the therapeutically effective amount of therapeutic agent to administer to the subject to treat the subject's at least one addictive disease, relative to the therapeutically effective amount of the therapeutic agent to administer to the standard suffering from the at least one addictive disease, wherein the first and second allele of the standard comprise the human serotonin 1B receptor gene comprising a DNA sequence of SEQ ID NO:1.

Furthermore, a method for determining a therapeutically effective amount of therapeutic agent to administer to a subject suffering from at least one addictive disease may further comprise an additional step of determining whether the second allele of the bodily sample taken from the subject comprises the human serotonin 1B receptor gene comprises a DNA sequence having at least one variation in SEQ ID NO:1, wherein the at least one variation comprises A−161T.

Such a variation in the first and/or second allele of the bodily sample is expected to be indicative of the therapeutically effective amount of the therapeutic agent to administer to the subject to treat the at least one addictive disease of the subject relative to the therapeutically effective amount of the therapeutic agent to administer to the standard suffering from the at least one addictive disease.

The presence of the human serotonin 1B receptor gene comprising a DNA sequence having at least one variation in SEQ ID NO:1 in the first and/or second alleles of the bodily sample taken from the subject, wherein the variation comprises A−161T is expected to be indicative of an altered therapeutically effective amount of the therapeutic agent to administer to the subject to treat the at least one addictive disease of the subject, relative to the therapeutically effective amount of the therapeutic agent to administer to the standard suffering from the at least one addictive disease, wherein the two alleles of the standard comprise the human serotonin 1B receptor gene comprising a DNA sequence of SEQ ID NO:1.

The present invention further extends to commercial test kits suitable for use by a medical professional to determine whether either or both alleles of a bodily sample taken from a subject comprise a DNA sequence having at least one variation in SEQ ID NO:1, wherein the variation comprises A−161T.

Commercial test kits of the present invention have applications in determining susceptibility of pain in the subject relative to a standard. Such kits can also be used to determine a subject's increased or decreased susceptibility to at least one addictive disease relative to susceptibility to at least one addictive disease in a standard. Also a therapeutically effective amount of pain reliever to administer to the subject in order to induce analgesia in the subject relative to a therapeutically effective amount of pain reliever to administer to a standard to induce analgesia in the standard can be determined. Moreover, a test kit of the present invention has applications in determining a therapeutically effective amount of therapeutic agent for treating at least one addictive disease to administer to a subject suffering from the at least one addictive disease, relative to a therapeutically effective amount of therapeutic agent to administer to a standard suffering from at least one addictive disease. The kit may also be used for the diagnosis or indication of susceptibility to a neurologic or psychiatric disorder, as described hereinabove. A standard as used herein comprises two alleles of the human serotonin 1B receptor gene comprising a DNA sequence of SEQ ID NO:1.

Furthermore, a commercial test kit of the present invention can also be used to determine the presence of an isolated variant allele of the human serotonin 1B receptor gene of the present invention in a bodily sample removed from a subject, which can serve as a genetic marker. As explained above, the predominant or “most common” allele of the human serotonin 1B receptor gene found in the population comprises a DNA sequence of SEQ ID NO:1. Hence a variant allele comprising a DNA sequence having a variation in SEQ ID NO:1, wherein the variation comprises A−161T, can be detected in the bodily sample with a commercial kit of the invention.

Other variant alleles of the human serotonin 1B receptor gene of the present invention can be detected with a commercial test kit of the present invention. For example, an isolated variant allele of the human serotonin 1B receptor gene detectable with a commercial kit of the present invention, comprises a DNA sequence having at least two variations in SEQ ID NO:1, wherein the variations comprise at least A−161T.

Accordingly, a commercial test kit may be prepared for determining the presence of at least one variation in the human serotonin 1B receptor gene of either or both alleles in a bodily sample taken from a subject, wherein the commercial test kit comprises:

a) PCR oligonucleotide primers suitable for detection of an allele comprising the human serotonin 1B receptor gene having a DNA sequence with a variation in SEQ ID NO:1;

b) other reagents; and

c) directions for use of the kit.

Accordingly, it is an object of the present invention to provide a heretofore unknown variation the DNA sequence of the human serotonin 1B receptor gene wherein the variation can be used to map the locus of the human serotonin 1B receptor gene.

It is yet another object of the present invention to use a heretofore unknown polymorphism of an allele of the human serotonin 1B receptor gene as a marker for any kind of disorder related to the human serotonin 1B receptor, such as an addictive disease, pain, a neurologic or psychiatric condition, or markers for genes.

It is another object of the present invention to provide nucleotides, optionally detectably labeled, hybridizable under standard hybridization conditions to variant alleles of the human serotonin 1B receptor gene disclosed herein.

It is another object of the present invention to gain insight into a subject's susceptibility to an addictive, neurologic or psychiatric condition or disease. This insight can be used to determine a therapeutically effective dose of pain reliever to administer to the subject to induce analgesia therein relative to the therapeutically effective amount of pain reliever administered to a standard to induce analgesia therein, wherein the standard comprises two alleles of the human serotonin 1B receptor gene comprising a DNA sequence of SEQ ID NO:1. Determining therapeutically-effective doses for neurologic and psychiatric disorders is also provided.

Such information can be used to tailor a regimen for treating a subject suffering from at least one addictive, neurologic or psychiatric disease, relative to the therapeutically effective amount of therapeutic agent administered to a standard suffering from at least one such disease.

It is yet another object of the present invention to provide commercial test kits for attending medical professionals to determine the presence of variant alleles of the human serotonin 1B receptor gene in a bodily sample taken from a subject. The results of such testing can then be used as a genetic marker to determine the subject's susceptibility to an addictive, neurologic or psychiatric condition or disease, susceptibility to at least one such disease, determining a therapeutically effective amount of pain reliever to administer to the subject in order to induce analgesia, or determining a therapeutically effective amount of therapeutic agent for treating at least one other such disease to administer to the subject.

It is yet another object of the present invention to provide commercial detecting variant alleles of the human serotonin 1B receptor gene in a bodily sample taken from a subject. The results of such tests can then be used to gain insight into a subject's ability to withstand pain, susceptibility to addiction, or to diagnose a neurologic or psychiatric disease or disorder.

These and other aspects of the present invention will be better appreciated by reference to the following drawings and Detailed Description.

### BRIEF DESCRIPTION OF THE DRAWINGS

FIG. 1: DNA sequence which shows the A−161T polymorphism of the human serotonin 1B receptor gene (SEQ ID No:2). The T at position −161 is identified in bold, and the start codon for the receptor protein is shown in bold.

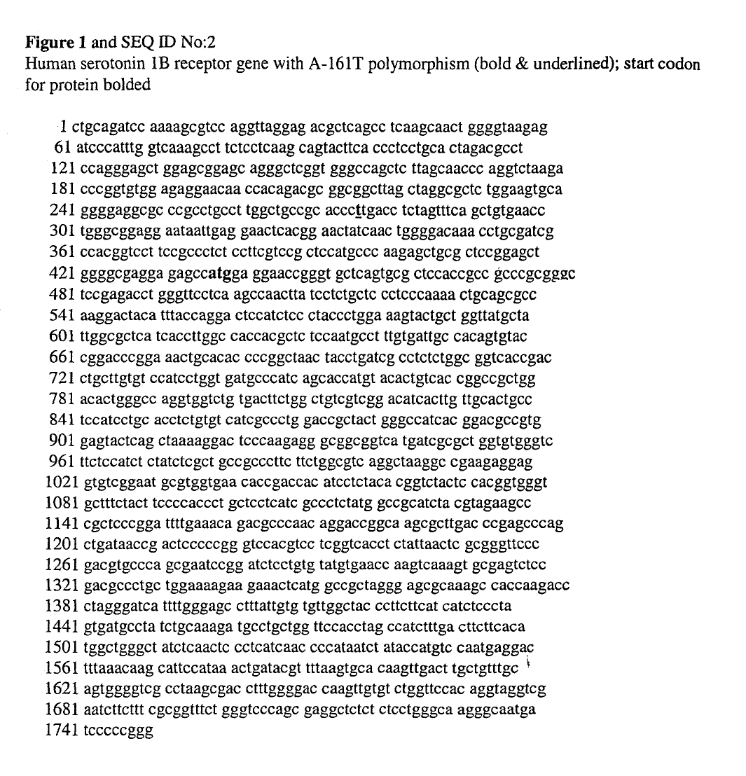

FIG. 2: Electropherogram of the serotonin 1B gene of an individual wild type at position −161.

FIG. 3: Electropherogram of the serotonin 1B gene of an individual heterozygous at position −161 with the A−161T polymorphism.

FIG. 4: Electropherogram of the serotonin 1B gene of an individual homozygous at position −161 for the A−161T polymorphism.

### DETAILED DESCRIPTION OF THE INVENTION

As explained above, the present invention is based upon Applicants' surprising and unexpected discovery of a heretofore unknown single nucleotide polymorphism (SNPs) in the human serotonin 1B receptor, wherein the A at position −161 is a T. Furthermore, Applicants have discovered that the SNP can be present in either (heterozygous) or both (homozygous) alleles of the human serotonin-1B receptor gene in a subject. The A−161T may also be present in combination with any of the previously-known serotonin 1B receptor gene SNPs.

Furthermore, the present invention is based upon Applicants' prediction that variant allele of the serotonin 1B receptor gene, which comprise a DNA sequence having a variation in SEQ ID NO:1, wherein the variation comprises A−161T, will result in altered expression of the serotonin 1B receptor gene product, and thus the presence of such variant allele in a bodily sample from a subject is expected to be indicative of the activity of the serotonin 1B receptor in the subject.

The present invention further extends to the heretofore unknown A−161T polymorphism of the human serotonin 1B receptor gene that can serve as genetic markers to map the locus of the human serotonin 1B receptor gene.

The present invention further extends to Applicants' discovery that the A−161T polymorphism is present in the population and that the presence of such polymorphism in the human serotonin 1B receptor gene of a subject may correlate with an increased or decreased susceptibility to addictive diseases, such as heroin addiction, cocaine addiction, or alcohol addiction, for example, and anxiety, depression, pathological aggression, and compulsive gambling, relative to such functions in a standard.

The present invention extends to diagnostic methods to determine a subject's increased or decreased susceptibility to at least one addictive or other neurologic or psychiatric disease. With the results of such methods, targeted prevention methods, early therapeutic intervention, and improved chronic treatment such as to opioid addiction are set forth herein and encompassed by the present invention. In addition, attending medical professionals of subjects armed with the results of such diagnostic methods can determine whether administration of opioid or other analgesics is appropriate or whether non-opioid derived analgesics should be administered to the subject. Also, appropriate choice and type of analgesic can be made in treating a subject's pain.

Also, the present invention extends to methods of determining a subject's increased or decreased susceptibility to an addictive, neurologic or psychiatric condition or disease and response to analgesics, and using that information when prescribing analgesics to the subject.

The present invention further extends to a variant allele of the human serotonin 1B receptor gene comprising a DNA sequence comprising a heretofore unknown polymorphism of A−161T. Furthermore, Applicants' invention extends to variant alleles of the human serotonin 1B receptor gene comprising a DNA sequence having at least two variations in the predominant or “most common” allele comprising the human serotonin 1B receptor gene comprising a DNA sequence of SEQ ID NO:1, wherein one of the variations comprises A−161T.

Consequently, an initial aspect of the present invention involves isolation of a heretofore unknown variant allele of the human serotonin 1B receptor gene. As used herein, the term “gene” refers to an assembly of nucleotides that encode a polypeptide, and includes cDNA and genomic DNA nucleic acids.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). Preferably a minimum length for a hybridizable nucleic acid is at least about 10 nucleotides; preferably at least about 20 nucleotides; and more preferably the length is at least about 30 nucleotides; and most preferably 40 nucleotides. One of skill in the art can determine for particular conditions the sequence and length of a nucleic acid, including a labeled nucleic acid, capable of selectively hybridizing to the variant allele of the invention and discriminating it from the wild-type allele.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C.

The present invention includes among various means for detecting the presence of the A−161T SNP, the use of molecular beacons, which are nucleic acid probes that recognizes and report the presence of a specific nucleic acid in homogeneous solutions. The probes are hairpin-shaped sequences with a central stretch of nucleotides complementary to the target sequence and termini comprising short mutually complementary sequences, one terminus covalently bound to a fluorophore and the other to a quenching moiety. When in their native state with hybridized termini, the proximity of the fluorophore and the quencher produce no fluorescence. The beacon undergoes a spontaneous fluorogenic conformational change when hybridized to its target nucleic acid.

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” or “promoter” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

A coding sequence is “operatively associated with” a transcriptional and translational control sequences, such as a promoter for example, when RNA polymerase transcribes the coding sequence into mRNA, which in turn is translated into a protein encoding by the coding sequence.

A “signal sequence” is included at the beginning of the coding sequence of a protein to be expressed on the surface of a cell. This sequence encodes a signal peptide, N-terminal to the mature polypeptide, that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached-to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The phrase,“expected to be indicative” is used herein to refer to the correlation between the identity of the allelic variation(s) in an individual and the susceptibility of an individual to addictive disease, as described above, or neurologic or psychiatric disease, such as but not limited to anxiety, depression, pathological aggression, and compulsive gambling. The correlations are based on the findings in the present invention of the relationship between the biochemistry and cellular function of the variants of the serotonin 1B receptor and clinical observations, analyzed statistically, on history of drug dependence and other previous or current conditions.

As explained above, within the scope of the present invention are DNA sequences encoding variant alleles of the human serotonin 1B receptor gene of the present invention, which comprise at least one variation in the predominant or “most common” allele of the human serotonin 1B receptor gene. The most common allele comprises a DNA sequence of SEQ ID NO:1, and a variation in the most common allele comprise A−161T.

In another embodiment, the present invention comprises DNA sequences encoding variant alleles of the human serotonin 1B receptor gene, comprising at least two variations in the predominant or “most common” allele of the human serotonin 1B receptor gene, wherein the most common human serotonin 1B receptor gene comprises a DNA sequence of SEQ ID NO:1. Variant alleles of the human serotonin 1B receptor gene encompassed by the present invention comprise a DNA sequence comprising at least two variations of SEQ ID NO:1, wherein one of the variations is A−161T.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667).

Accordingly, the term “sequence similarity” in all its grammatical forms refers to the degree of identity or correspondence between nucleic acid or amino acid sequences of proteins that do not share a common evolutionary origin (see Reeck et al., supra). However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” is used herein to refer to similar or homologous sequences, whether the exact position is identical or different from the molecule to which the similarity or homology is measured. Thus, the term “corresponding to” refers to the sequence similarity, and not the numbering of the amino acid residues or nucleotide bases.

A variant allele of the human serotonin 1B receptor gene of the present invention, whether genomic DNA or cDNA, can be isolated from any source, particularly from a human cDNA or genomic library. Methods for obtaining an allele of the human serotonin 1B receptor gene, variants thereof, or the most common, are well known in the art, as described above (see, e.g., Sambrook et al., 1989, supra).

Accordingly, any human cell can potentially serve as the nucleic acid source for the molecular cloning of a variant allele of the human serotonin 1B receptor gene of the present invention, or a nucleic acid molecule hybridizable to a variant allele of the human serotonin 1B receptor gene of the present invention. The DNA may be obtained by standard procedures known in the art from cloned DNA (e.g., a DNA “library”), and preferably is obtained from a cDNA library prepared from tissues with high level expression of the human serotonin 1B receptor protein, by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, an allele of the human serotonin 1B receptor gene of the present invention should be molecularly cloned into a suitable vector for propagation.

In the molecular cloning of the human serotonin 1B receptor gene of the present invention, DNA fragments are generated, some of which will encode an allele. The DNA may be cleaved at specific sites using various restriction enzymes. Alternatively, one may use DNAse in the presence of manganese to fragment the DNA, or the DNA can be physically sheared, as for example, by sonication. The linear DNA fragments can then be separated according to size by standard techniques, including but not limited to, agarose and polyacrylamide gel electrophoresis and column chromatography.

Once the DNA fragments are generated, identification of the specific DNA fragment containing an allele of the human serotonin 1B receptor of the present invention may be accomplished in a number of ways. For example, if an amount of a portion of an allele of the human serotonin 1B receptor gene, or its specific RNA, or a fragment thereof, is available and can be purified and labeled, the generated DNA fragments may be screened by nucleic acid hybridization to the labeled probe (Benton and Davis, 1977, Science 196:180; Grunstein and Hogness, 1975, Proc. Natl. Acad. Sci. U.S.A. 72:3961).

A labeled cDNA of an allele of the human serotonin 1B receptor gene of the present invention, or fragments thereof, or a nucleic acid hybridizable under standard hybridization conditions to an allele of the human serotonin 1B receptor gene of the present invention, can be synthesized using sequences set forth herein. The radiolabeled mRNA or cDNA may then be used as a probe to identify homologous DNA fragments from among other genomic DNA fragments. Suitable labels include enzymes, radioactive isotopes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g., U.V. light to promote fluorescence. Among examples of colored labels, which can be used according to the present invention, include metallic sol particles, for example, gold sol particles such as those described by Leuvering (U.S. Pat. No. 4,313,734); dye sol particles such as described by Gribnau et al. (U.S. Pat. No. 4,373,932) and May et al. (WO 88/08534); dyed latex such as described by May, supra, Snyder (EP-a 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419-439, 1980 and in U.S. Pat. No. 4,857,453.

Other labels for use in the invention include magnetic beads or magnetic resonance imaging labels.

In addition, a molecular beacon capable of identifying the SNP herein may be used to determine the presence of the gene variant in a sample, including whether the individual is heterozygous or homozygous for the variant.

### Cloning and Expression Vectors

The present invention also relates to cloning vectors comprising variant alleles of the human serotonin 1B receptor gene of the present invention, and an origin of replication. For purposes of this Application, an “origin of replication” refers to those DNA sequences that participate in DNA synthesis.

As explained above, in an embodiment of the present invention, variant alleles of the human serotonin 1B receptor gene of the present invention comprise a DNA sequence having at least one variation in the most common allele of the human serotonin 1B receptor gene comprising a DNA sequence of SEQ ID NO:1, wherein the variation comprises A−161T.

In another embodiment, the present invention extends to variant alleles of the human serotonin 1B receptor gene, comprising a DNA sequence having at least two variations in the DNA sequence of SEQ ID NO:1, wherein one of the variations is A−161T.

Furthermore, an isolated variant allele of the human serotonin 1B receptor gene of the present invention, or isolated nucleic acid molecules hybridizable under standard hybridization conditions to an isolated variant allele of the human serotonin 1B receptor gene of the present invention, can be inserted into an appropriate cloning vector in order to produce multiple copies of the variant allele or isolated nucleic acid molecule. A large number of vector-host systems known in the art may be used. Possible vectors include, but are not limited to, plasmids or modified viruses. The vector system used however must be compatible with the host cell used. Examples of vectors include having applications herein, but are not limited to E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating a variant allele of the human serotonin 1B receptor gene of the present invention, or an isolated nucleic acid hybridizable thereto under standard hybridization conditions, into a cloning vector which has complementary cohesive termini. However, if the complementary restriction sites used to fragment the variant allele or isolated nucleic acid hybridizable thereto are not present in the cloning vector, the ends of the variant allele or the isolated nucleic acid molecule hybridizable thereto under standard hybridization conditions may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Such recombinant molecules can then be introduced into host cells via transformation, transfection, infection, electroporation, etc., so that many copies of a variant allele of the human serotonin 1B receptor gene of the present invention, or an isolated nucleic acid molecule hybridizable thereto under standard hybridization conditions, can be generated. Preferably, the cloned isolated variant is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

In an alternative method an isolated variant allele of the human serotonin 1B receptor gene of the present invention or an isolated nucleic acid molecule hybridizable thereto under standard hybridization conditions may be identified and isolated after insertion into a suitable cloning vector in a “shot gun” approach. Enrichment for a variant allele, for example, by size fractionation, can be done before insertion into the cloning vector.

As the A−161T SNP is in a non-coding region of the serotonin receptor gene, its presence may alter the level of expression of the gene product. Therefore, the present invention extends to expression systems which include the non-coding region of the gene, and the ability to evaluate the extent of expression which is conferred on the expressed portion of the gene by virtue of the presence of the A−161T variation, in combination with one or more of the other known serotonin 1B receptor gene polymorphisms.

Consequently, the present invention extends to a method for determining a susceptibility of a subject to one addictive disease, neurologic or psychiatric disease or disorder comprising removing a bodily sample comprising a first and second allele of the human serotonin 1B receptor gene from the subject, and determining whether either the first or second alleles, or both alleles comprise a DNA sequence having at least one variation in SEQ ID NO:1, wherein the variation comprises A−161T.

The presence of the variation of A−161T in either or both alleles of the human serotonin 1B receptor gene of a sample from the subject indicates the subject has an altered susceptibility to at least one addictive disease relative to a standard having alleles of the human serotonin 1B receptor gene comprising a DNA sequence of SEQ ID NO:1.

Furthermore, the presence of the variation of comprising A−161T, in either or both alleles of a serotonin 1B receptor gene in a sample from the subject is expected to indicate the subject as a altered susceptibility to addictive diseases relative to a standard comprising alleles of the human serotonin 1B receptor gene comprising a DNA sequence of SEQ ID NO:1.

In this embodiment, the biological sample can be a biological fluid, such as but not limited to, blood, or a biopsy sample such as a scraping of cells from the inside of the cheek. Alternatively, variant alleles of the human serotonin 1B receptor gene indicating increased or decrease susceptibility to addictive diseases in the subject as described above, can be detected from cellular sources, such as, but not limited to, brain tissue biopsies, adipocytes, testes, heart, and the like. For example, cells can be obtained from an individual by biopsy and lysed, e.g., by freeze-thaw cycling, or treatment with a mild cytolytic detergent such as, but not limited to, TRITON X-100®, digitonin, NONIDET P (NP)-40®, saponin, and the like, or combinations thereof (see, e.g., International Patent Publication WO 92/08981, published May 29, 1992). In yet another embodiment, samples containing both cells and body fluids can be used (see ibid.).

Other methods presently understood by a skilled artisan, and encompassed by the present invention, can also be used to detect the presence of either variation in either or both alleles of the human serotonin 1B receptor gene in a sample, and hence increased or decreased susceptibility to at least one addictive disease of the subject relative to the susceptibility of at least one addictive disease in a standard comprising alleles of the human serotonin 1B receptor gene comprising a DNA sequence of SEQ ID NO:1.

For example, an optionally detectably labeled isolated nucleic acid molecule hybridizable under standard hybridization conditions to an isolated variant allele of the human serotonin 1B receptor gene comprising a DNA sequence having a variation in SEQ ID NO:1, wherein the variation comprises A−161T, can be used in standard Northern hybridization analysis to detect the presence, and in some instances quantitate the level of transcription of such a variant allele of the present invention. The presence of this variant allele in a bodily sample from a subject is expected to be indicative of increased susceptibility to at least one addictive disease in the subject. Likewise, an optionally detectably labeled isolated nucleic acid molecule hybridizable under standard hybridization conditions to an allele of the human serotonin 1B receptor gene comprising a DNA sequence having a variation in SEQ ID NO:1, wherein the variation comprises A−161T can be used in a standard Northern hybridization analysis to detect the presence of a variant allele in the sample comprising a variation in SEQ ID NO:1, wherein the variation comprises A−161T, which is expected to be indicative of a decreased susceptibility to at least one addictive disease relative to the susceptibility of a standard comprising two alleles of the human serotonin 1B receptor gene comprising a DNA sequence of SEQ ID NO:1.

Alternatively, oligonucleotides of the invention can be used as PCR primers to amplify an allele of the human serotonin 1B receptor gene of the biological sample e.g., by reverse transcriptase-PCR, or amplification of the allele itself. The amplified mRNA or DNA can then be quantified or sequenced in order to determine the presence of a variant allele, and the susceptibility of the subject to addictive diseases. Furthermore, variations in SEQ ID NO:1, as described above, can be found by creation or deletion of restriction fragment length polymorphisms (RFLPs) not found in the predominant or “most common” allele, hybridization with a specific probe engineered to hybridize to variation described above under standard hybridization conditions, (or lack of hybridization with a probe specific for the predominant or “most common” allele), as well as by other techniques.

Numerous methods presently available, and understood by the skilled artisan, can be used to “genotype” a subject in regards to the presence of a variant allele of the human serotonin 1B receptor gene in the genome of the subject. In particular, methods described above to ascertain increased or decreased susceptibility to addictive diseases have relevance in this embodiment of the present invention, and can readily be used herein. For example, Northern blot hybridization an isolated nucleic acid of the present invention hybridizable under standard hybridization conditions to an isolated variant allele of the human serotonin 1B receptor gene comprising a DNA sequence having a variation of SEQ ID NO:1, wherein the variation comprises A−161T, as a probe, along with RT-PCR, PCR, and numerous immunoassays described above, have applications herein.

### Commercial Kits

Furthermore, as explained above, the present invention extends to commercial kits having applications in screening a bodily sample taken from a subject for the presence of at least the variant allele comprising the human serotonin 1B receptor comprising a DNA sequence having a variation in SEQ ID NO:1, wherein the variation comprises A−161T.

With information obtained from the use of a test kit of the present invention, an attending health profession can determine whether the subject has a susceptibility to addictive, neurologic or psychiatric diseases relative to a standard, an increased susceptibility relative to the susceptibility of a standard, a therapeutically effective amount of agents directed toward the particular condition or disease.

Accordingly, a test kit of the present invention for determining whether a subject comprises a variant allele of the human serotonin 1B receptor gene comprising a DNA sequence having a variation in SEQ ID NO:1, comprises means for detecting the presence of a variation in a first and or second allele comprising the human serotonin 1B receptor in a biological sample from a subject, and optimally packaged with directions for use of the kit. In one particular aspect, the means for detecting the presence of a variant allele of the human serotonin 1B receptor gene comprising a DNA sequence having the A−161T variation in SEQ ID NO:1, a test kit comprises an oligonucleotide probe for binding to a variant allele of the human serotonin 1B receptor gene comprising a DNA sequence having a variation in SEQ ID NO:1; and means for detecting the level of binding of the probe to the variant allele, wherein detection binding of the probe to the variant allele indicates the presence of a variant comprising the human serotonin 1B receptor gene comprising a DNA sequence having a variation in SEQ ID NO:1.

The sequence of the oligonucleotide probe used in a commercial kit will determine which if any such variation is present in an allele comprising the human serotonin 1B receptor gene. Should no binding be detected, it is probable that no such variation exists in either allele of the subject.

More specifically, a commercial test kit of the present invention comprises a) PCR oligonucleotide primers suitable for detection of a variant allele of the human serotonin 1B receptor gene comprising a DNA sequence having a variation in SEQ ID NO:1, i.e., A−161T, b) other reagents; and c) directions for use of the kit.

Examples of PCR oligonucleotide primer suitable for detection of an allele comprising the human serotonin 1B receptor gene comprising a DNA sequence having a variation in SEQ ID NO:1 can be readily produced by a person of ordinary skill in the art with teaching set forth herein., and variations of SEQ ID NO:1 also set forth herein.

The present invention may be better understood by reference to the following non-limiting Example, which is provided as exemplary of the invention. The following Example is presented in order to more fully illustrate the preferred embodiments of the invention. It should in no way be construed, however, as limiting the broad scope of the invention.

### EXAMPLE

Molecular cloning of the serotonin 1B receptor has made it possible to identify potential sequence polymorphisms. To identify unknown SNPs of the serotonin 1B receptor, a PCR-based strategy was used to amplify the coding regions of the receptor gene, and to determine the DNA sequence of the amplified exon. Using this method DNA samples were sequenced from 157 subjects.

The sequences were determined by evaluation of electropherograms obtained by automated sequencing of a PCR amplicon of the serotonin 1B receptor gene amplified by PCR from human genomic DNA using primers specific for the serotonin 1B gene. The primers used were: forward primer (−595) 5′ CAG CGC TGC TCC TAG ACT TC3′(SEQ ID No:3) and reverse primers (1247) 5′ TTC GAC CTA CCT GTG GAA CC3′ (SEQ ID No:4) or (1339) 5′ GCT CTC TCT CAG GAC TAT TCT3′ (SEQ ID No:5).

The gene from each sample was sequenced in both the forward and reverse direction with the following sequencing primers: Forward (−595) 5′ CAG CGC TGC TCC TAG ACT TC3′ (SEQ ID No:6); and Reverse (−40) 5′ ATG GAG CGG ACG AAG GAG A3′ (SEQ ID No:7).

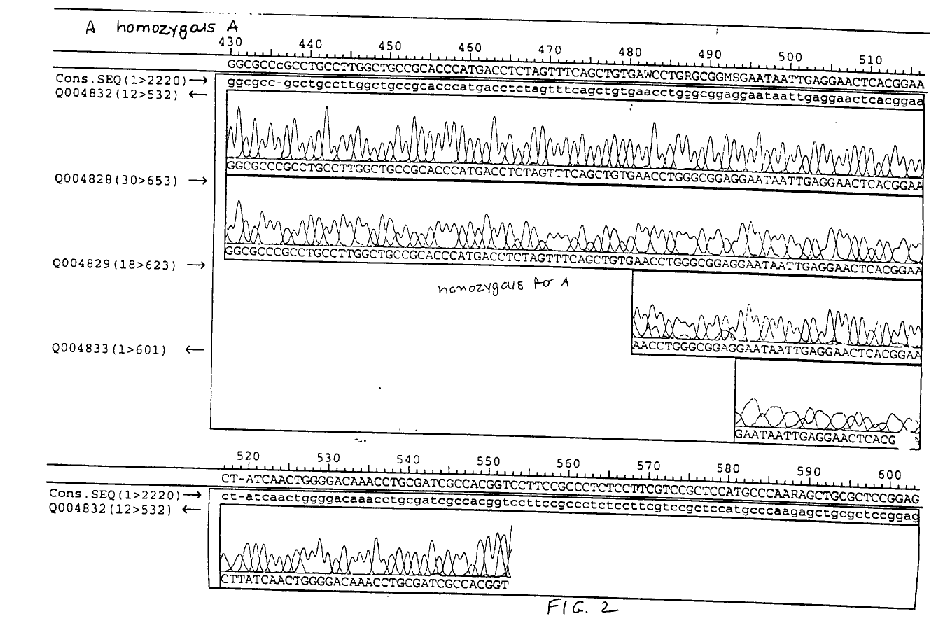

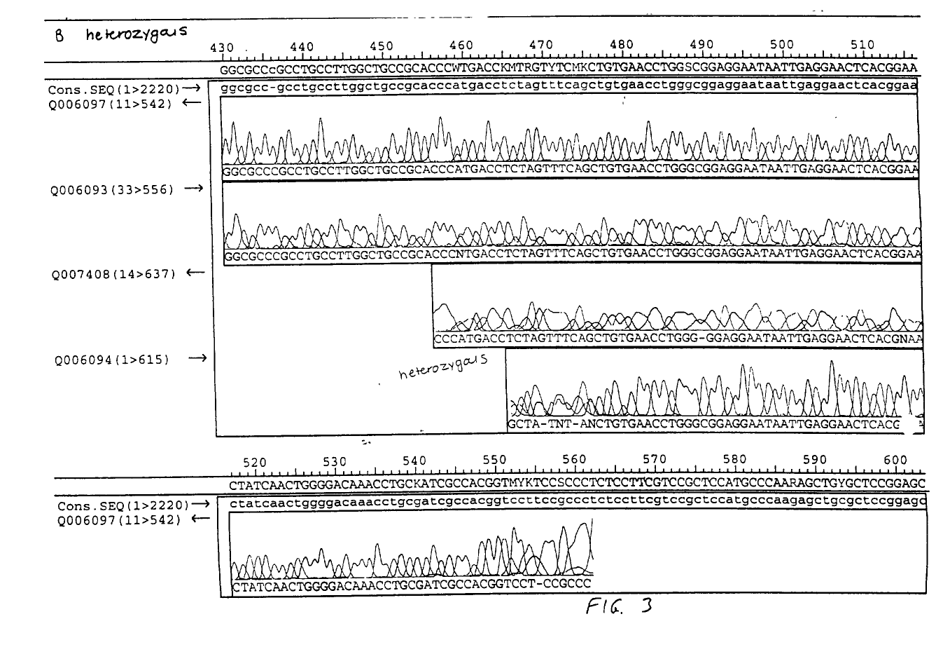

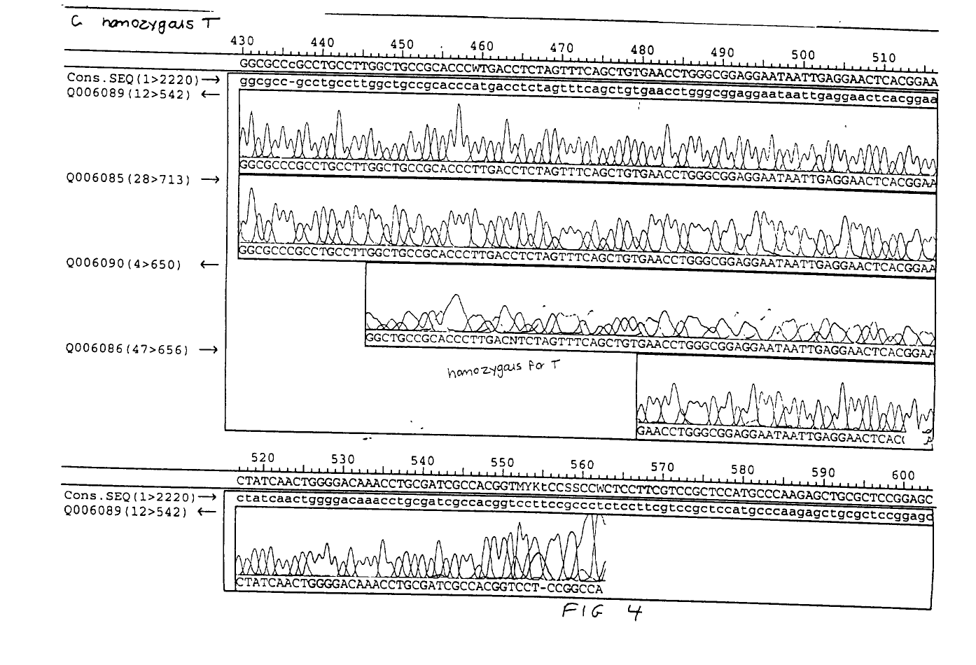

As shown in the electropherogram of the wild-type individual (FIG. 2), only A is present at position −161. In contrast, the electropherogram from a heterozygous individual (FIG. 3), position −161 has both an A and T present; in the homozygous individual (FIG. 4), only T is present.

By sequencing PCR-amplified DNA from the study subjects, it was determined that the previously reported sequence for the human serotonin 1B receptor (SEQ ID No: 1) was the most common allele found in the study population. Several already-known SNPs were also identified. For the purpose of this study, the term “most common” was used to denote the predominant serotonin 1B receptor allele and the corresponding receptor that was originally reported by cDNA cloning (SEQ ID No: 1), and the term “variant” to denote the allelic genes/receptors containing polymorphic variations, such as A−161T.

Of 314 alleles studied from 157 individuals, 61 alleles had the A−161T SNP. Five of the 157 individuals were homozygous for the A−161T polymorphism.

Various publications are cited throughout the Specification, of which all are incorporated herein by reference in their entireties.

### References

1. Reisine, T. & Pasternak, G. W. (1996) in Goodman & Gilman's The Pharmacological Basis of Therapeutics, (eds. Hardman, J. G., Limbird, L. E., Molinoff, P. B., Ruddon, R. W. & Gilman, A. G.) 9th ed., 521-554 McGraw-Hill, New York, 1996.

2. Kreek, M. J. Opiates, opioids and addiction. Mol. Psychiatry 1, 232-254 (1996).

3. Pasternak, G. W. Pharmacological Mechanisms of opioid analgesics. Clin. Neuropharmacol. 16, 1-18 (1993).

4. Yu, L. In Encyclopedia of Human Biology (ed Dulbecco, R.) 2nd ed., 445-450 Academic Press, (1997).

5. Zadina, J. E., Hackler, L., Ge, L. J., & Kastin, A. J. A potent and selective endogenous agonist for the mu-opiate receptor. Nature 386, 499-502 (1997).

6. Kreek, M. J. Opioid receptors: some perspectives from early studies of their role in normal physiology, stress responsivity, and in specific addictive diseases. Neurochem. Res. 21, 1469-1488 (1996).

7. Chen, Y., Mestek, A., Liu, J., Hurley, J. A. & Yu, L. Molecular cloning and functional expression of a mu-opioid receptor from rat brain. Mol. Pharmacol. 44, 8-12 (1996).

8. Wang, J. B., Johnson, P. S., Persico, A. M., Hawkins, A. L., Griffin, C. A. & Uhl, G. R. Human mu opioid receptor: cDNA and genomic clones, pharmacologic characterization and chromosomal assignment. FEBS Lett. 338, 217-222 (1994).

9. Mestak, A., Hurley, J. H., Bye, L. S., et al. The human serotonin 1B receptor: Modulation of functional desensitization by calcium/calmodulin-dependent protein kinase and protein kinase C. J. Neurosci. 15, 2396-2406 (1995).

10. Berrettini, W. H., Hoehe, M. R., Ferrada, T. N. & Gottheil, E. (1997) Addiction Biol. 2, 303-308.

11. Kreek, M. J. Medical complications in methadone patients. Ann. N.Y. Acad. Sci., 311, 110-134 (1978).

12. Kreek, M. J. Using methadone effectively: achieving goals by application of laboratory, clinical, and evaluation research and development of innovative programs. NIDA Res. Monograph, 106, 245-266 (1991).

13. Borg, L., Broe, D. M., Ho, A. & Kreek, M. J. in Problems of Drug Dependence 1994: Proceedings of the 56th Annual Scientific Meeting of the College on Problems of Drug Dependence (ed Harris, L. S.) 17 (1995).

14. Woody, G. E., McLellan, A. T., Luborsky, L., O'Brien, C. P., Blaine, J., Fox, S., Herman, I. & Beck, A. T. (1984) Am. J. Psychiatry 141, 1172-1177.

15. Mason, B., Kreek, M. J., Kocsis, J., Melia, D. & Sweeney, J. (1992) in Problems of Drug Dependence 1991: Proceeding of the 53rd Annual Scientific Meeting of the College on Problems of Drug Dependence, ed. Harris, L. S. 230.

16. Mantel, N. & Haenszel, W. (1959) J. Natl. Cancer Inst. 22, 719-748.

17. O'Dowd, B. F., Lefkowitz, R. J. & Caron, M. G. (1989) Ann. Rev. Neurosci. 12, 67-83.

18. Chen, Y., Mestek, A., Liu, J. & Yu, L. (1993) Biochem. J. 295, 625-628.

19. North, R. A. (1993) in Handbook of Experimental Pharmacology (vol. 104): Opioids I, ed. Herz, A. (Springer-Verlag, Berlin), pp. 773-797.

20. Chavkin, C. (1988) in The Opiate Receptors, ed. Pasternak, G. W. (Humana Press, New Jersey), pp. 273-303.

21. Kubo, Y., Reuveny, E., Slesinger, P. A., Jan, Y. N. & Jan, L. Y. (1993) Nature 364, 802-806.

22. Dascal, N., Schreibmayer, W., Lim, N. F., Wang, W., Chavkin, C., DiMagno, L., Labarca, C., Kieffer, B. L., Gaveriaux-Ruff, C., Trollinger, D., Lester, H. A. & Davidson, N. (1993) Proc. Natl. Acad. Sci. USA 90, 10235-10239.

23. Chen, Y. & Yu, L. (1994) J. Biol. Chem. 269, 7839-7842.

24. Kovoor, A., Henry, D. J. & Chavkin, C. (1995) J. Biol. Chem. 270, 589-595.

25. Kreek, M. J. & Hartman, N. (1982) Ann. N. Y Acad. Sci. 398, 151-172.

26. Herz, A. (1993) Opioids I & II (Springer-Verlag, Berlin).

27. Zhou, Y., Spangler, R., LaForge, K. S., Maggos, C. E., Ho, A. & Kreqek, M. J. (1996) Peptides 17, 435-441.

28. Kreek, M. J., Wardlaw, S. L., Friedman, J., Schneider, B. & Frantz, A. G. (1981) in Advances in Endogenous and Exogenous Opioids, eds. Simon, E. & Takagi, H. (Kodansha Ltd. Publishers, Tokyo, Japan), pp. 364-366.

29. Kreek, M. J., Wardlaw, S. L., Hartman, N., Raghunath, J., Friedman, J., Schneider, B. & Frantz, A. G. (1983) Life Sci. 33 Suppl 1, 409-411.

30. Kreek, M. J., Ragunath, J., Plevy, S., Hamer, D., Schneider, B. & Hartman, N. (1984) Neuropeptides 5, 277-278.

31. Ragavan, V. V., Wardlaw, S. L., Kreek, M. J. & Frantz, A. G. (1983) Neuroendocrinology 37, 266-268.

32. Kosten, T. R., Kreek, M. J., Swift, C., Carney, M. K. & Ferdinands, L. (1987) Life Sci. 41, 1071-1076.

33. Kosten, T. R., Kreek, M. J., Ragunath, J. & Kleber, H. D. (1986) Life Sci. 39, 55-59.

34. Kennedy, J. A., Hartman, N., Sbriglio, R., Khuri, E. & Kreek, M. J. (1990) Br. J. Addiction 85, 1133-1140.

35. Culpepper-Morgan, J. A., Twist, D. J., Petrillo, C. R., Soda, K. M. & Kreek, M. J. (1992) Metabolism: Clinical & Experimental 41, 578-581.

36. Culpepper-Morgan, J. A. & Kreek, M. J. (1997) Metabolism: Clinical & Experimental 46, 130-134.

37. Kreek, M. J. (1997) Pharmacol. Biochem. Behav. 57, 551-569.

39. McLellan, A. T., Luborsky, L., Woody, G. E. & O'Brien, C. P. (1980) J. Nerv. Ment. Dis. 168, 26-33.

40. Bare, L. A., Mansson, E. & Yang, D. (1994) FEBS Lett. 354, 213-216.

41. Chen, Y., Liu, J. & Yu, L. (1996) Addiction Biol. 1, 49-59.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. An isolated variant allele of the human serotonin 1B receptor gene, comprising a DNA sequence having a variation in SEQ ID NO:1, wherein said variation comprises A−161T.

2. The isolated variant allele of claim 1, detectably labeled.

3. The isolated variant allele of claim 2, wherein said detectable label comprises a radioactive element, a chemical which fluoresces, or an enzyme.

4. An isolated nucleic acid molecule hybridizable to said isolated variant allele of claim 1 under standard hybridization conditions.

5. The isolated nucleic acid molecule of claim 4, detectably labeled.

6. The isolated nucleic acid molecule of claim 5, wherein said detectable label comprises a radioactive element, a chemical which fluoresces, or an enzyme.

7. A cloning vector comprising an isolated variant allele of the human serotonin 1B receptor gene and an origin of replication, wherein said variant allele comprises a DNA sequence having a variation in SEQ ID NO:1, wherein said variation comprises A−161T.

8. A cloning vector comprising an origin of replication and an isolated nucleic acid molecule hybridizable under standard hybridization conditions to an isolated variant allele of the human serotonin 1B receptor gene, wherein said variant allele comprises a DNA sequence having at least one variation in SEQ ID NO:1, wherein said at least one variation comprises A−161T.

9. The cloning vector of either of claims 7 or 8, wherein said cloning vector comprises of E. coli, bacteriophages, plasmids, or pUC plasmid derivatives.

10. The cloning vector of claim 9, wherein bacteriophages further comprise lambda derivatives, plasmids further comprise pBR322 derivatives, and pUC plasmid derivatives further comprise pGEX vectors, or pmal-c, pFLAG.

11. An expression vector comprising an isolated variant allele of the human serotonin 1B receptor gene comprising a DNA sequence having a variation in SEQ ID NO:1, wherein said variation comprises A−161T.

12. An expression vector comprising an isolated nucleic acid molecule hybridizable under standard hybridization conditions to an isolated variant allele of the human serotonin 1B receptor gene, wherein said isolated nucleic acid molecule is operatively associated with a promoter, and said variant allele comprises a DNA sequence having at least one variation in SEQ ID NO:1, wherein said at least one variation comprises A−161T.

13. The expression vector of either of claims 11 or 12, wherein said promoter comprises immediate early promoters of hCMV, early promoters of SV40, early promoters of adenovirus, early promoters of vaccinia, early promoters of polyoma, late promoters of SV40, late promoters of adenovirus, late promoters of vaccinia, late promoters of polyoma, the lac the trp system, the TAC system, the TRC system, the major operator and promoter regions of phage lambda, control regions of fd coat protein, 3-phosphoglycerate kinase promoter, acid phosphatase promoter, or promoters of yeast α mating factor.

13. A unicellular host transformed or transfected with an expression vector comprising an isolated variant allele of the human serotonin 1B receptor gene operatively associated with a promoter, wherein said variant allele comprises a DNA sequence having at least one variation in SEQ ID NO:1, wherein said at least one variation comprises A−161T.

14. A unicellular host transformed with an expression vector comprising an isolated nucleic acid molecule hybridizable under standard hybridization conditions to an isolated variant allele of the human serotonin 1B receptor gene, wherein said isolated nucleic acid molecule is operatively associated with a promoter, and said variant allele comprises a DNA sequence having at least one variation in SEQ ID NO:1, wherein said at least one variation comprises A−161T.

15. The unicellular host of either of claims 13 or 14, wherein said host comprises E. coli, Pseudomonas, Bacillus, Streptomyces, yeast, CHO, R1.1, B-W, L-M, COS1, COS7, BSC1, BSC40, BMT10 or Sf9 cells.

16. A method for determining a susceptibility in a subject to at least one addictive disease, comprising the steps of:
a) removing a bodily sample from said subject, wherein said sample comprises a first and second allele comprising the human serotonin 1B receptor gene;
b) determining whether said human serotonin 1B receptor gene of said first allele comprises a DNA sequence having at least one variation in SEQ ID NO:1, wherein said variation comprises A−161T;
such that the presence of said at least one variation in said human serotonin 1B receptor gene of said first allele is expected to be indicative of the subject's susceptibility to at least one addictive disease relative to the susceptibility to said at least one addictive disease in a standard.

17. The method for determining a susceptibility to at least one addictive disease of claim 16, further comprising the step of determining whether said human serotonin 1B receptor gene of said second allele comprises a DNA sequence having at least one variation in SEQ ID NO:1, wherein said variation comprises A−161T, such that the presence of said at least one variation in said human serotonin 1B receptor gene of said second allele is expected to be indicative of the subject's susceptibility to said at least one addictive disease relative to the susceptibility to said at least one addictive disease in said standard.

18. The method of either of claims 16 or 17, wherein said at least one variation of said human serotonin 1B receptor gene of said first and/or second allele comprises A−161T., and said variation is expected to be indicative of a altered susceptibility to said at least one addictive disease in the subject relative to the susceptibility to said at least one addictive disease in the standard.

19. The method of either of claims 16 or 17, wherein said at least one addictive disease comprises: opioid addiction; cocaine addiction or addiction to other psychostimulants; nicotine addiction; barbiturate or sedative hypnotic addiction; anxiolytic addiction; or alcohol addiction.

20. The method of either of claims 16 or 17, wherein where said at least one variation of said human serotonin 1B receptor gene of said first and/or second allele comprises A−161T, and said variation is expected to be indicative of an increased susceptibility to said at least one addictive disease in said subject relative to susceptibility to said at least one addictive disease in said standard.

21. The method of claim 20, wherein said at least addictive disease comprises: opioid addiction; cocaine addiction or addiction to other psychostimulants; nicotine addiction; barbiturate or sedative hypnotic addiction; anxiolytic addiction; or alcohol addiction.

22. A method for determining a susceptibility into a subject, neurologic or psychiatric condition or disease, wherein the method comprises the steps of:
a) removing a bodily sample from said subject, wherein said sample comprises a first and second allele comprising the human serotonin 1B receptor gene;
b) determining whether said human serotonin 1B receptor gene of said first allele comprises a DNA sequence having at least one variation in SEQ ID NO:1, wherein said variation comprises A−161T;
such that the presence of said at least one variation in said human serotonin 1B receptor gene of said first allele is expected to be indicative of susceptibility to a neurologic or psychiatric condition or disease in said subject relative to susceptibility to a neurologic or psychiatric condition or disease in said standard, wherein said first allele of said standard-comprises the human serotonin 1B receptor gene comprising a DNA sequence of SEQ ID NO:1.

23. The method of claim 22 for determining a susceptibility to a neurologic or psychiatric condition or disease in a subject, further comprising the step of determining whether said second allele of said bodily sample comprises the human serotonin 1B receptor gene comprising a DNA sequence having at least one variation in SEQ ID NO:1, wherein said variation comprises A−161T; such that the presence of said at least one variation in said second allele is expected to be indicative of susceptibility to a neurologic or psychiatric condition or disease in said subject relative to susceptibility of pain in said standard, wherein said second allele of said standard comprises the human serotonin 1B receptor gene comprising a DNA sequence of SEQ ID NO:1.

24. The method of either of claims 22 or 23, wherein where said at least one variation in said human serotonin 1B receptor gene of said first and/or second allele of said bodily sample from said subject comprises A−161T, wherein said variation is expected to be indicative of an altered susceptibility to a neurologic or psychiatric condition or disease in said subject relative to susceptibility to a neurologic or psychiatric condition. or disease in said standard.

25. The method of claims 22-24 wherein said neurologic or psychiatric diseases is anxiety, depression, pathological aggression, or compulsive gambling.

26. A method for determining a therapeutically effective amount of pain reliever to administer to said subject, comprising the steps of:
a) removing a bodily sample from said subject, wherein said sample comprises a first and second allele comprising the human serotonin 1B receptor gene; and
b) determining whether said first allele comprises the human serotonin 1B receptor gene comprising a DNA sequence having at least one variation in SEQ ID NO:1, wherein said at least one variation comprises A−161T;
wherein the presence of said at least one variation in said human serotonin 1B receptor gene of said first allele is expected to be indicative of the subject's susceptibility to pain relative to said to susceptibility of pain in said standard, wherein said first allele of said standard comprises the human serotonin 1B receptor gene comprising a DNA sequence of SEQ ID NO:1, such that said therapeutically effective amount of pain reliever to administer to the subject in order to induce analgesia is related to said susceptibility to pain in said subject relative to susceptibility to pain.

27. The method of claim 26, wherein determining susceptibility to an addictive, neurologic or psychiatric condition or disease in said subject relative to susceptibility to an addictive, neurologic or psychiatric condition or disease in said standard further comprises the step of determining whether said second allele of said bodily sample from said subject comprises the human serotonin 1B receptor gene comprising a DNA sequence having at least one variation in SEQ ID NO:1, wherein said at least one variation comprises A−161T;
such that the presence of said at least one variation in said second allele is expected to be indicative of susceptibility to an addictive, neurologic or psychiatric condition or disease in said subject relative to susceptibility to an addictive, neurologic or psychiatric condition or disease in said standard, wherein said second allele of said standard comprises the human serotonin 1B receptor gene comprising a DNA sequence of SEQ ID NO:1, and the therapeutically effective amount of pain reliever to administer to said subject to induce analgesia in said subject is related to the presence of said at least one variation in said human serotonin 1B receptor gene of said second allele of said bodily sample from said subject.

28. A commercial test kit may for determining the presence of at least one variation in the human serotonin 1B receptor gene of an allele in a bodily sample taken from a subject, wherein the commercial test kit comprises:
a) PCR oligonucleotide primers suitable for detection of an allele comprising the human serotonin 1B receptor gene comprising a DNA sequence having a variation in SEQ ID NO:1 which is A−161T;
b) other reagents; and
c) directions for use of the kit.
